(19) United States
(12) Patent Application Publication
(10) Pub. No.: US 2014/0088513 A1
(43) Pub. Date: Mar. 27, 2014

(54) MODULAR GAS-ACTUATED RETRACTABLE NEEDLE ASSEMBLY

(71) Applicants: Brett Leigh Darichuk, Calgary (CA); Lionel Stuart Matthews, Calgary (CA)

(72) Inventors: Brett Leigh Darichuk, Calgary (CA); Lionel Stuart Matthews, Calgary (CA)

(73) Assignee: L.O.M. LABORATORIES INC., Calgary, AB (CA)

(21) Appl. No.: 14/118,536

(22) PCT Filed: Mar. 26, 2012
(86) PCT No.: PCT/CA2012/050184

§ 371 Date: Nov. 18, 2013

Related U.S. Application Data
(60) Provisional application No. 61/491,768, filed on May 31, 2011.

(51) Int. Cl. A61M 5/32

(52) U.S. Cl. CPC A61M 5/3234
USPC 604/198

(57) ABSTRACT

A modular gas-actuated retractable needle assembly, syringes for engagement with the modular gas-actuated retractable needle assembly, and kits comprising same are provided. In some embodiments, the syringe has a distal swedge to facilitate engagement of syringes of a plurality of different sizes with a single modular gas-actuated retractable needle assembly. Methods of using the modular gas-actuated retractable needle assembly or a syringe assembly having a modular gas-actuated retractable needle assembly are provided.

### REFERENCE TO RELATED APPLICATIONS

For purposes of the United States of America, this application claims the benefit of U.S. provisional patent application No. 61/491,768 filed 31 May 2011, which is hereby incorporated by reference herein in its entirety for all purposes.

### TECHNICAL FIELD

This application relates to retractable needle assemblies, and in particular to retractable needle assemblies for gas-actuated retractable syringes.

### BACKGROUND

It is well known that many dangerous communicable diseases are spread through contacting the body fluids of an infected person. After use of a syringe, residual body fluids are likely to remain on or within the syringe needle. For this reason, syringes are typically intended for a single use only. In order to be handled safely after use, the needle of a syringe must be covered to prevent it from accidentally stabbing a person who is, for example, collecting the syringe for disposal, thereby releasing residual body fluids into such person. Typically, a protective cap is provided with the syringe, which after use of the syringe can be used to cover the tip of the needle. However, it sometimes happens that persons attempting to cap a used needle miss the cap and accidentally stab themselves, resulting in potential exposure to communicable diseases.

Accordingly, it is desirable to provide a syringe wherein the needle can be retracted into the syringe following use. Syringes including retractable needles wherein the retraction of the needle is accomplished by means of pneumatic actuation have been developed, as exemplified by U.S. Pat. No. 5,868,713 to Klippenstein and U.S. Pat. No. 7,811,259 to Klippenstein, both of which are incorporated by reference herein.

There remains a need for retractable syringe designs which reduce manufacturing costs and permit greater flexibility in the choice of syringe and/or needle size (e.g. gauge and/or length).

The foregoing examples of the related art and limitations related thereto are intended to be illustrative and not exclusive. Other limitations of the related art will become apparent to those of skill in the art upon a reading of the specification and a study of the drawings.

### SUMMARY

A modular gas-actuated retractable syringe assembly has a syringe for receiving a retractable needle, the syringe having a barrel, a medicament chamber within the barrel, and a plunger. The plunger is slidably disposed within the syringe barrel to displace medicament from the medicament chamber. A modular gas-actuated retractable needle assembly is engageable with the syringe so that the needle is placed in fluid communication with the medicament chamber to administer the medicament. The modular gas-actuated retractable needle assembly has a gas release cell and a gas cell puncturer disposed within a gas release chamber. The application of a post-injection force to the plunger by a user causes the gas cell puncturer to rupture the gas cell and the pressure of the released gas within the gas release chamber forces the needle to be retracted within the syringe.

In some embodiments, the needle has a needle header at a proximal end thereof and the plunger has a needle port seal at a distal end thereof, and the needle port seal is engageable with the needle header to provide a needle carrier upon the application of the post injection force. In some embodiments, the syringe has a swedge of larger diameter than the other portions of the syringe barrel at its distal end.

In some embodiments, a modular gas-actuated retractable needle assembly for coupling to a syringe so that the needle is placed in fluid communication with a medicament chamber of the syringe to administer medicament is provided. The syringe has a plunger for administering medicament with a retraction lumen therein and a needle carrier for engaging with and retracting the needle under gas pressure. The assembly includes a mating surface for engaging with the syringe, a needle releasably secured within the assembly, a gas release chamber defined between a distal portion of the assembly and the needle, a gas release cell disposed within the gas release chamber, and a gas cell perforator disposed within the gas release chamber and operable in response to the application of a post-injection force to rupture the gas release cell. When in use, a post-injection force applied by a user to the plunger couples the needle to the needle carrier and causes the gas cell perforator to puncture the gas release cell.

In some embodiments, a syringe for use with a modular gas-actuated retractable needle assembly having a needle with a needle header at a proximal end of the needle, a gas release cell and a gas release trigger mechanism with a gas release chamber of the modular gas-actuated retractable needle assembly is provided. The syringe includes a barrel having a first diameter, a distal swedge having a diameter greater than the first diameter, a plunger axially slidable and sealingly engaged within the barrel of the syringe, the plunger having a retraction lumen therein, a needle port seal initially sealingly engaged within the plunger at a distal end of the retraction lumen, and a medicament chamber defined within the barrel distally of the plunger, the medicament chamber being configured to be placed in fluid communication with the needle when the syringe is assembled with the modular gas-actuated retractable needle assembly. In use when assembled with the modular gas-actuated retractable needle assembly and upon the application of a post-injection force to the plunger by a user, the needle port seal engages with the needle header and the gas release trigger mechanism ruptures the gas release cell so that the needle port seal is displaced axially in the proximal direction with respect to the retraction lumen to retract the needle within the retraction lumen.

In some embodiments, a kit including a plurality of syringes of different volumes for engagement to a single modular retractable needle assembly is provided. Each of the syringes includes a barrel, a plunger axially slidable and sealingly engaged within the barrel of the syringe for administering medicament, the plunger having a retraction lumen therein, a needle port seal sealingly engaged within the plunger at a distal end of the retraction lumen, and a medicament chamber defined within the barrel distally of the plunger. The modular retractable needle assembly includes a mating surface for engaging with the syringe, a needle releasably secured within the assembly for being placed in fluid communication with the medicament chamber, a gas release chamber defined between a distal portion of the assembly and the needle, a gas release cell disposed within the gas release chamber, and a gas cell perforator disposed within the gas release chamber and operable in response to the application of a post-injection force to rupture the gas release cell. In use, application of a post-injection force to the plunger by a user couples the needle to the needle carrier and causes the gas cell perforator to puncture the gas release cell to retract the needle into the retraction lumen of the plunger.

### BRIEF DESCRIPTION OF DRAWINGS

Exemplary embodiments are illustrated in referenced figures of the drawings. It is intended that the embodiments and figures disclosed herein are to be considered illustrative rather than restrictive.

FIG. 1 is an exploded view showing the basic component parts of a retractable syringe assembly according to one embodiment of the invention.

FIG. 2 is a partially exploded partially sectional view of the embodiment of FIG. 1 showing the retractable needle assembly engaged with the syringe.

FIG. 3A is an exploded perspective view and FIG. 3B is a perspective view of the modular retractable needle assembly of the embodiment of FIG. 1.

FIG. 4A is an exploded sectional view and FIG. 4B is a sectional view of the modular retractable needle assembly of the embodiment of FIG. 1.

FIG. 5 is a partially sectional view of the embodiment of FIG. 1 showing the modular retractable needle assembly coupled to the syringe and the plunger engaged within the syringe in a partially withdrawn position. Enlarged sectional views showing the main components of the modular needle assembly and the engagement of the needle port seal within the plunger retraction lumen are shown.

FIG. 6 is a partially sectional view of the embodiment of FIG. 1 showing the plunger at the end of the injection phase. An enlarged sectional view showing the engagement between the needle port seal and the needle header is shown.

FIG. 7 is a partially sectional view of the embodiment of FIG. 1 showing the plunger almost at the end of the post-injection phase. An enlarged sectional view showing the main components of the modular retractable needle assembly is shown.

FIG. 8 is a sectional view of the embodiment of FIG. 1 showing the needle in the retracted state within the plunger lumen.

FIG. 9 shows a plurality of syringes of different sizes shaped and configured for engagement with a single modular retractable needle assembly according to one embodiment of the invention.

FIG. 10 shows a sectional view of the syringes of FIG. 9 with correspondingly sized and shaped plungers engaged therein.

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

In this specification, “seals” or “sealingly engages” means that two elements are engaged with sufficient sealing capability that the function for which the sealing is provided can be effectively performed.

“Distal” means the direction towards the tip of the needle when the hypodermic syringe assembly is in the assembled state. “Proximal” means the direction opposite of distal, i.e. the direction away from the tip of the needle when the hypodermic syringe assembly is in the assembled state.

“Injection force” means a force that would typically be applied by a user to the plunger of a syringe to inject a medicament into a patient.

“Post-injection force” means a force that may be slightly greater than an injection force that is applied to activate the gas-actuated retraction mechanism described below after a user has completed injection of the medicament.

“Loading force” means a force typically applied by a user when drawing medicament into a syringe in preparation for administering that medicament to a patient.

Although the invention is described with reference to an exemplary embodiment of a modular assembly for a retractable hypodermic needle and syringe, embodiments of the invention have application in other devices where needles are used with patients, for example intravenous needle assemblies.

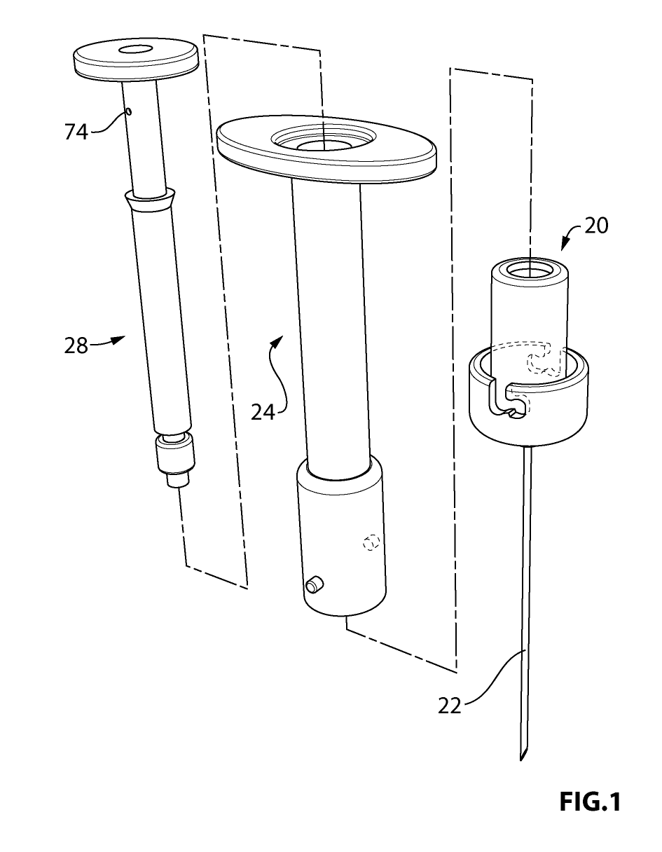

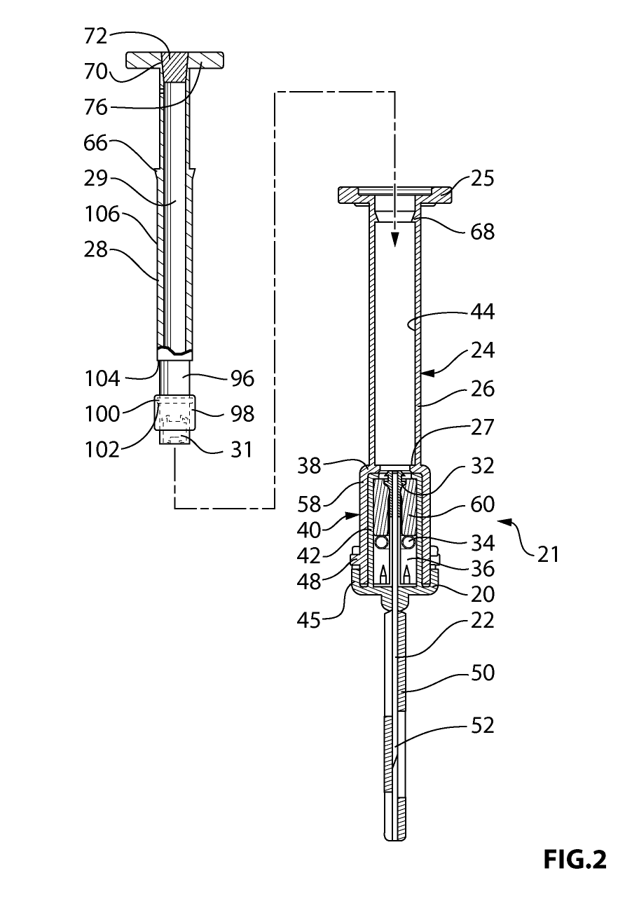

With reference to FIG. 1, in one embodiment a retractable needle assembly 20 having a hypodermic needle 22 may be coupled to a syringe 24 for use in administration of a medicament to a patient using a plunger 28. As shown in FIG. 2, syringe 24 has a barrel 26, a plunger 28 slidingly and sealingly engaged within the barrel 26 for applying pressure and/or suction to needle 22, and a modular retractable needle assembly 20 coupled to the distal end of the syringe barrel 26. The overall assembly of retractable needle assembly 20, syringe 24, and plunger 28 provides a syringe assembly 21.

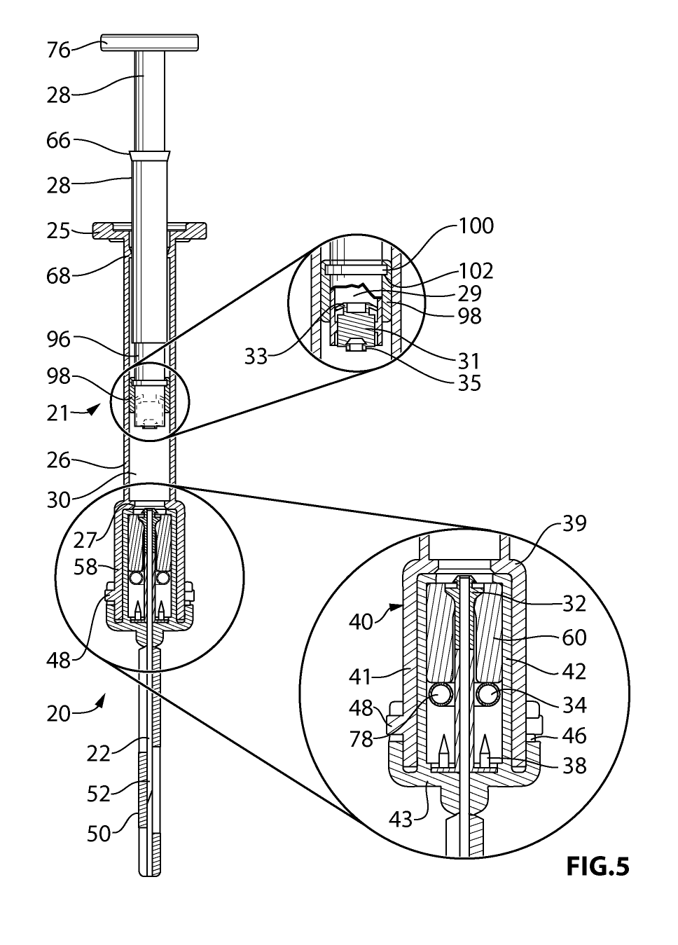

A medicament chamber 30 (FIG. 5) is defined between modular retractable needle assembly 20, the distal end of plunger 28 and an interior surface 44 of syringe barrel 26 for containing the medicament to be administered to the subject. Medicament chamber 30 is in fluid communication with needle 22 when syringe assembly 21 is in the assembled configuration.

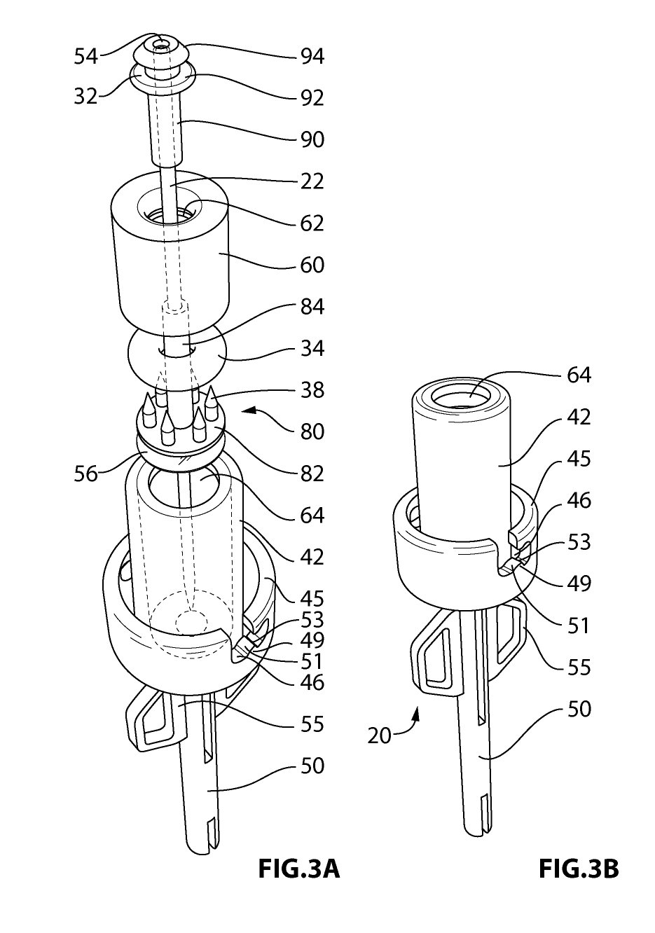

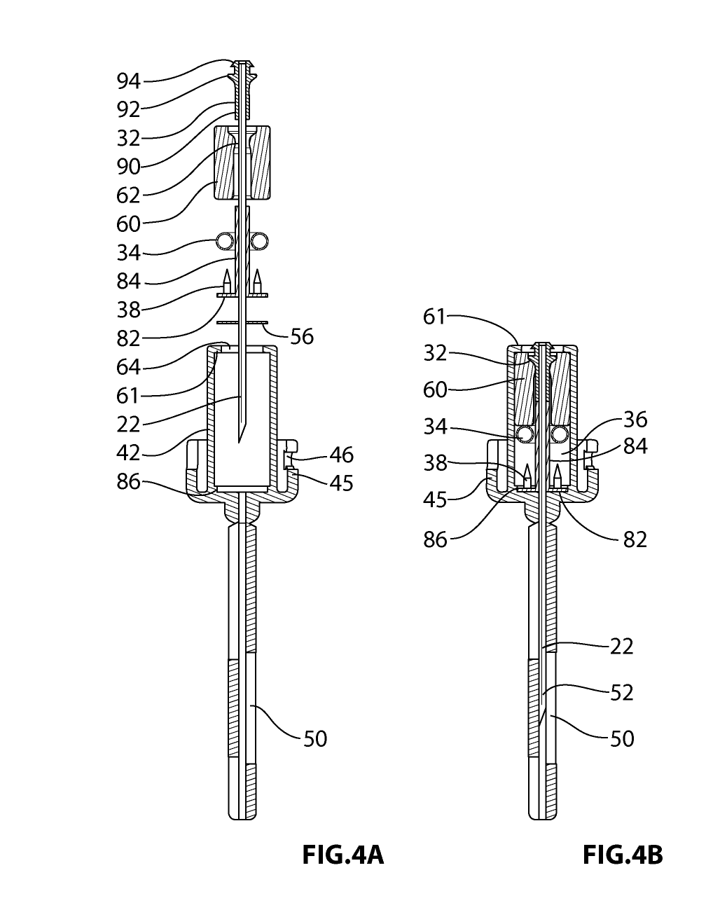

With reference to FIGS. 3A, 3B, 4A and 4B, modular retractable needle assembly 20 includes a needle 22 integrally formed with or affixed to a needle header 32, a gas release cell 34 within a gas release chamber 36 (FIG. 4B), and a rupturing mechanism 38 for rupturing gas release cell 34 upon application of a post-injection force by a user.

In some embodiments of the present invention, the force exerted by the release of a propellant causes both the needle header and the plunger to move proximally within syringe barrel 26, e.g. in the manner described in U.S. Pat. No. 5,868,713 to Klippenstein.

In other embodiments of the present invention, including the illustrated embodiment, the plunger may be locked at or near the distal-most point of its travel and the needle is received within the plunger. With reference to FIG. 5, to facilitate needle retraction in this manner, plunger 28 includes a retraction lumen 29 (FIG. 2) therewithin for receiving needle 22 when needle 22 is retracted and a needle port seal 31 at a distal end of plunger 28 for assisting in the retraction of needle 22 by engaging with a needle header 32 in the manner described below.

Syringe 24 preferably includes a flange 25 formed therewith or attached thereto to facilitate grasping and usage of syringe assembly 21 by a user. Flange 25 may be provided at or near the proximal end of syringe 24. Flange 25 may be any suitable shape, for example a generally circular extension projecting radially outwardly from the barrel of syringe 24 or a pair of opposed projecting tabs serving as finger grips.

In some embodiments, including the illustrated embodiment, syringe 24 includes an internal radial wall 27 therein (FIG. 2). Internal radial wall 27 is provided towards the distal end of syringe 24 to stop movement of plunger 28 after injection of medicament into a subject in the manner described below.

Syringe 24 may include a distal portion of relatively larger diameter than the rest of barrel 26, hereafter referred to as a “swedge”, to facilitate engagement of a plurality of different sizes of syringe 24 to a single modular retractable needle assembly 20 as described below. In the illustrated embodiment, syringe 24 includes a swedge 40 at its distal end. Swedge 40 includes a proximal wall portion 39 extending radially outwardly from syringe barrel 26 and a distally projecting cylindrical extension 41 of wider diameter than the rest of barrel 26 (FIG. 5).

Modular retractable needle assembly 20 has a mating surface for securing assembly 20 to a syringe 24. In the illustrated embodiment, the mating surface is provided by the exterior surface of a cylindrical axial extension 42 (FIGS. 3A and 4A), which is of sufficient diameter to engage with interior surfaces of a correspondingly shaped mating receptacle 58 at the distal end of syringe barrel 26. Cylindrical axial extension 42 extends in the proximal direction from a base 43 of assembly 20. Any suitable sealing engagement may be used to couple modular retractable needle assembly 20 to syringe 24, including for example a threaded engagement, or a Luer-Lok™ or Luer-Slip™ fitting, as are commonly used to couple conventional hypodermic needles to syringes.

Assembly 20 may further include a securing feature for securely coupling assembly 20 to syringe 24 so that assembly 20 is not readily separated from syringe 24. In the illustrated embodiment, a pair of L-shaped slots 46 (FIGS. 3A and 3B) are provided on an exterior securing jacket 45 on assembly 20. Securing jacket 45 is a generally cylindrical extension that projects in the proximal direction from base 43. Securing jacket 45 is spaced radially apart from cylindrical axial extension 42 so that mating receptacle 58 on the distal end of syringe 24 can be received between extension 42 and securing jacket 45.

In some embodiments, syringe 24 is sealingly engaged to retractable needle assembly 20. In some embodiments, a suitable seal can be provided between modular retractable needle assembly 20 and syringe 24. In some embodiments, the seal is an O-ring seal that sits between the distal tip of syringe 24 and base 43 of assembly 20.

Slots 46 are shaped and positioned to engage with a securing projection 48 on the outside surface of mating receptacle 58 of syringe 24. In the illustrated embodiment, securing projections 48 project radially outwardly from the barrel 26 of syringe 24. Together, L-shaped slots 46 and securing projections 48 provide a securing feature that prevents relative movement of assembly 20 and syringe 24 in an axial direction when assembly 20 is in the secured position. The number of engaging slots and projections could be varied, and the position of the slots and projections need not be symmetric, although assembly of modular retractable needle assembly 20 with syringe 24 will be facilitated in embodiments where the slots and projections are symmetrically disposed about the circumference of assembly 20 and syringe 24, respectively.

In embodiments in which the modular retractable needle assembly is secured to the syringe via a Luer-Lok™ fitting or via a threaded engagement, the securing feature may be provided by the lip of the female portion of the Luer-Lok™ fitting (not shown), or by the engagement of the threads on the modular retractable needle assembly and the syringe, respectively. In some embodiments, such as those where the modular retractable needle assembly is coupled to the syringe by a Luer-Slip™ fitting, no securing feature is present. In such embodiments, frictional forces are sufficient to keep modular retractable needle assembly 20 coupled to the syringe 24 during normal use.

In some embodiments, modular retractable needle assembly 20 is detachably coupled to syringe 24. In other embodiments, including the illustrated embodiment, a one-way locking mechanism is provided, so that once modular retractable needle assembly 20 has been coupled to syringe 24 it will be very difficult to remove assembly 20 from syringe 24. The one-way locking mechanism helps to avoid tampering or risk of injury that could arise if assembly 20 were to be removed from syringe 24 after needle 22 has been used and retracted.

In the illustrated embodiment, a one-way locking protrusion is provided within L-shaped slots 46 to provide a one-way locking mechanism. In the illustrated embodiment, one-way locking protrusion 49 (FIGS. 3A and 3B) has a first surface, angled surface 51, that is angled, which allows securing projection 48 to be slid past one-way locking protrusion 49 within slot 46. Angled surface 51 is provided on the side of one-way locking protrusion 49 past which securing projection 48 must be slid to place syringe assembly 21 in the locked configuration. One-way locking protrusion 49 has a second surface, securing surface 53, which is axial, nearly axial, or in some other way configured to prevent motion of securing projections 48 in a direction that would allow assembly 20 to be removed from syringe 24. Securing surface 53 is provided on the side of one-way locking protrusion 49 that is directed toward securing projection 48 when securing projection 48 is in the locked configuration. Thus, one-way locking protrusion 49 allows assembly 20 to be easily secured to syringe 24, but makes removal of assembly 20 very difficult once assembly 20 has been placed in the locked configuration.

Assembly 20 includes needle 22 projecting from its distal end. Needle 22 is securely but releasably retained within assembly 20 so that needle 22 is securely retained in place when syringe 24 is in normal use, i.e. during loading of medicament into medicament chamber 30 and during injection of medicament into a subject. Needle 22 is releasable (via release of needle header 32 as described below) in response to force applied by the rupture of gas release cell 34 so that needle 22 can be retracted into the body of syringe 24 upon the application of a post-injection force by a user, as described below.

A needle cover 50 is provided to cover needle 22 prior to use. Needle cover 50 may be laminated, cemented or otherwise affixed to modular retractable needle assembly 20 to secure it in place prior to use. Needle cover 50 can be twisted off or otherwise removed in any suitable manner to expose needle 22 for use. Needle cover 50 may include radially extending tabs 55 or other surface features to facilitate removal of needle cover 50.

In the illustrated embodiment, needle 22 is releasably retained in the resting position via needle header 32, as described below. Needle header 32 is provided at or near the proximal end of needle 22. Needle header 32 may be formed integrally with or separately from needle 22. In some embodiments, needle 22 may be crimped in, cemented to, or otherwise securely fixed to needle header 32. Needle header 32 securely retains needle 22 in place against the distally applied force of medicament being injected into a patient or against a proximal loading force, but is releasable in the proximal direction in response to pressure produced by the release of a propellant from a gas release cell 34 as described below.

In the illustrated embodiment, needle header 32 engages with needle port seal 31 (provided in the distal tip of plunger 28 as described below) on or shortly before the application of a post-injection force by a user. Needle header 32 and/or needle port seal 31 provide a distal bearing surface for the application of proximally directed force created by gas pressure upon rupture of gas release cell 34. Needle port seal 31, alone or in combination with needle header 32, acts as a needle carrier to pull needle 22 into retraction lumen 29 through the force applied by compressed propellant released from gas release cell 34.

Needle 22 is hollow and has a downstream tip 52 for injection of medicament into a subject and an upstream intake opening 54 (FIG. 3A) for receiving medicament from medicament chamber 30. In some embodiments, a needle membrane 56 is provided to cover the distal end of modular assembly 20 to more tightly seal gas release chamber 36 by sealingly engaging against needle 22.

Needle membrane 56 may be secured to the base 82 of perforator assembly 86 in any suitable manner, for example by suitable adhesives. Needle membrane 56 may assist in retaining needle 22 within syringe 24 once needle 22 has been retracted by providing a barrier to needle re-emergence. Needle membrane 56 may help to prevent any medicament from dripping off the end of needle 22 and into the surrounding environment after use.

Needle membrane 56 may be made of a soft, flexible material. In some embodiments, needle membrane 56 is made from a soft surgical-grade rubber such that, when needle 22 is retracted, needle membrane 56 tends to flow into itself and seal the hole left by needle 22. In some embodiments, needle membrane 56 is formed with a small, slightly conical hole that has a taper of approximately 30° relative to the longitudinal axis of syringe 24 positioned just at the point where needle 22 passes through needle membrane 56.

With particular reference to FIGS. 3A and 4B, a gas release cell 34 is contained within assembly 20 distally of needle header 32, within gas release chamber 36. In the illustrated embodiment, gas release chamber 36 is defined between the interior surface of cylindrical axial extension 42, the base 43 of assembly 20, and needle header 32. However, depending on the configuration of the components of syringe assembly 21, the gas release chamber 36 could be defined between other components of the assembly. For example, in embodiments in which perforator assembly 80 described below is sealingly engaged by perforator mount 86, gas release chamber 36 may be defined between the interior surface of cylindrical axial extension 42, the needle header 32, and the base 82 of perforator assembly 80.

Needle membrane 56 may optionally assist in the sealing of gas release chamber 36 to prevent the escape of compressed propellant when gas release cell 34 is ruptured. In some embodiments, the sealing between the components that define gas release chamber 36 is sufficiently tight to generally seal gas release chamber 36 and facilitate retraction of needle 22 without the need for additional sealing components such as needle membrane 56 and/or an O-ring disposed between the distal tip of syringe 24 and modular retractable needle assembly 20.

In the illustrated embodiment, needle header 32 is retained in place against the distal force applied by a user during the injection phase (an “injection force”) by an engagement ring 60. Engagement ring 60 has a central opening 62 through which needle 22 passes. In some embodiments, central opening 62 is cylindrically shaped. Engagement ring 60 is frictionally but slidably engaged with the interior surface of cylindrical extension 42. Engagement ring 60 is axially slidable within cylindrical extension 42 in response to the application of a post-injection force, but is retained in place within assembly 20 during the application of an injection force. Engagement ring 60 is also retained in place within assembly 20 during the application of a force in the proximal direction required to load medicament into medicament chamber 30 (i.e. a loading force).

In some embodiments, engagement ring 60 is frictionally secured in cylindrical extension 42. Engagement ring 60 could be secured in place in any suitable manner that is sufficiently strong to retain engagement ring 60 in place during the application of an injection or loading force, but releasable in response to the application of a post-injection force. For example, engagement ring 60 could be held in place by breakable tabs or weakly secured with an adhesive. In the illustrated embodiment, engagement ring 60 assists in puncturing gas release cell 34 in response to the application of a post-injection force, as described below.

In some embodiments, an internal radial wall 61 is provided on the internal surface of cylindrical extension 42 at or near its proximal end. Internal radial wall 61 may assist in ensuring that engagement ring 60 is not displaced within modular retractable needle assembly 20 prior to use. Internal radial wall 61 has a central opening 64 that is of a sufficiently large diameter to allow distal plunger neck 96 to enter into modular retractable needle assembly 20. In some embodiments, internal radial wall 27 may be absent from syringe 24, and internal radial wall 61 may perform the same functions as internal radial wall 27. In some embodiments, both internal radial wall 27 and internal radial wall 61 may not be present.

Engagement ring 60 allows needle header 32 (and thus needle 22) to move proximally into syringe 24 when gas release cell 34 is ruptured. Needle header 32 is also secured with engagement ring 60 in a manner that is sufficiently strong to ensure that needle header 32 does not separate from engagement ring 60 until after the rupture of gas release cell 34. For example, some loading force may be exerted against needle header 32 in the proximal direction when medicament is being loaded into syringe 24, and an injection force will be applied against needle header 32 in the distal direction when the medicament is injected. The engagement between needle header 32 and engagement ring 60 should be sufficiently strong so as not to separate during the application of a loading force in the proximal direction, but sufficiently weak that needle header 32 can separate from engagement ring 60 when gas release cell 34 is ruptured. Needle header 32 can be secured to engagement ring 60 in any suitable manner that provides such a releasable engagement, such as by sufficiently strong friction fit, sufficiently weak adhesive to allow release when gas release cell 34 is ruptured, easily frangible connectors, easily releasable connectors, or the like. In the illustrated embodiment, needle header 32 is secured within engagement ring 60 by a compression fit. In some embodiments, needle header 32 may be sized to be of sufficiently large diameter that needle header 32 cannot physically pass through engagement ring 60 in the distal direction.

In some embodiments, plunger 28 and syringe barrel 26 include a plunger locking feature to retain plunger 28 in a position at or near the distal limit of travel of plunger 28. In the illustrated embodiment, the plunger locking feature is provided by a plunger verge 66 and syringe barrel lock 68, which are positioned and configured to allow one-way sliding motion therebetween.

Plunger verge 66 is a radially outwardly extending circumferential protrusion on the outside surface of plunger 28. Syringe barrel lock 68 is a radially inwardly extending circumferential protrusion on the inside surface of syringe barrel 26. Both plunger verge 66 and syringe barrel lock 68 are sized so as not to interfere with the axial sliding motion between plunger 28 and syringe 24. However, plunger verge 66 and syringe barrel lock 68 are dimensioned and positioned so that after plunger verge 66 has been slid distally past syringe barrel lock 68, plunger verge 66 cannot thereafter be slid in a proximal direction past syringe barrel lock 68. This configuration essentially locks plunger 28 in a position at or near its distal-most point of travel, making it very difficult to separate plunger 28 from syringe 24 after use. This ensures needle 22 remains securely within retraction lumen 29 after use of syringe 24.

To achieve a one-way sliding motion, the distal surface of plunger verge 66 and/or the proximal surface of syringe barrel lock 68 may be angled or otherwise shaped so that plunger verge 66 may be readily slid past syringe barrel lock 68 in the distal direction. That is, plunger verge 66 may have an inclined surface progressively increasing in diameter from its distal-most portion and/or syringe barrel lock 68 may have an inclined surface progressively increasing in diameter from its proximal-most portion. This configuration allows plunger verge 66 to be easily slid past syringe barrel lock 68 in the distal direction. The proximal surface of plunger verge 66 and the distal surface of syringe barrel lock 68 may be flat, i.e. parallel or nearly parallel to a notional radial cross-section of syringe barrel 26, so that plunger verge 66 cannot readily be slid past syringe barrel lock 68 in the proximal direction.

Plunger 28 may optionally include an opening 70 at its proximal end (FIG. 2). Opening 70 may receive a plunger plug 72 in sealing engagement therein, so that needle 22 can be retained in retraction lumen 29 when needle 22 has been retracted.

In some embodiments, plunger 28 includes an orifice such as vent hole 74 therein (FIG. 1), for example on the proximal or side surface of plunger 28. Vent hole 74 may allow release of air from retraction lumen 29 upstream of needle header 32 and needle port seal 31 when needle 22 is retracted. Vent hole 74 should be positioned proximally of the upstream limit of travel of needle port seal 31, to avoid a loss of pressure that could stop the upstream travel of needle 22 before it has been fully retracted as could occur if, for example, vent hole 74 is positioned distally of the upstream limit of travel of needle port seal 31.

In some embodiments, the fit between plunger plug 72 and opening 70 is sufficiently tight to retain plunger plug 72 within opening 70, but is not airtight, so that air can be released upstream of needle header 32 when needle 22 is retracted.

Plunger 28 may also include a plunger end flange 76 to provide a bearing surface for the fingers of a user, e.g. to facilitate withdrawal of plunger 28 from syringe barrel 26 to draw liquid into medicament chamber 30 and/or administration of medicament using syringe assembly 21.

In the illustrated embodiment, gas release cell 34 is an annular gas cell dimensioned and configured to fit within gas release chamber 36. Gas release cell 34 has a central inner opening through which needle 22 passes, and contains a suitable non-toxic compressed propellant in its interior gas chamber 78. In some embodiments, the propellant initially comprises a liquid phase in its compressed state, and the propellant expands to a gas phase upon rupture of gas release cell 34. In some embodiments, the propellant initially comprises a compressed gas that expands upon rupture of gas release cell 34.

In some embodiments, gas release cell 34 has a generally rectangular shape, and may be bent or curved within gas release chamber 36 (not shown) such that the gas release cell takes on a curved shape. In some such embodiments, gas release cell 34 is bent or curved within gas release chamber 36 to have a generally circular shape, with a first edge of the gas release cell contacting or nearly contacting the opposite edge of the gas release cell within gas release chamber 36.

Gas release cell 34 may be initially secured within gas release chamber 36 in any suitable manner to minimize the risk that gas release cell 34 may be prematurely ruptured by rupturing mechanism 38. For example, gas release cell 34 may be frictionally engaged with the inner surface of cylindrical axial extension 42 or with perforator neck 84 (described below), or gas release cell 34 may be affixed to engagement ring 60, perforator neck 84, or cylindrical axial extension 42 in any suitable manner, such as by adhesives.

The pressure and volume of propellant in gas release cell 34 should be sufficient to ensure that needle 22 is fully retracted within retraction lumen 29 when gas release cell 34 is ruptured. Gas release cells intended for use with a larger volume of syringe may have a larger volume (and thus contain more compressed propellant) than gas release cells intended for use with a smaller volume of syringe. The appropriate pressure and volume of propellant to be included in gas release cell 34 can be determined by one skilled in the art based on the propellant to be used and the anticipated range of temperatures at which syringe assembly 21 will be used.

A mechanism for rupturing gas release cell 34 in response to a post-injection force is provided within gas release chamber 36. In the illustrated embodiment, a gas release trigger in the form of one or more puncture lances 38 is provided. Puncture lances 38 are secured within modular retractable needle assembly 20 in any suitable manner. In the illustrated embodiment, puncture lances 38 are secured to a perforator mount 80 secured at a distal portion of modular retractable needle assembly 20. Puncture lances 38 could alternatively be mounted to appropriate portions of engagement ring 60, or to the distal end of plunger 28 (with corresponding holes provided through engagement ring 60 to receive the puncture lances), or integrally formed with such components, such that puncture lances 38 are positioned and disposed to be operable to puncture gas release cell 34 in response to application of a post-injection force, as described below.

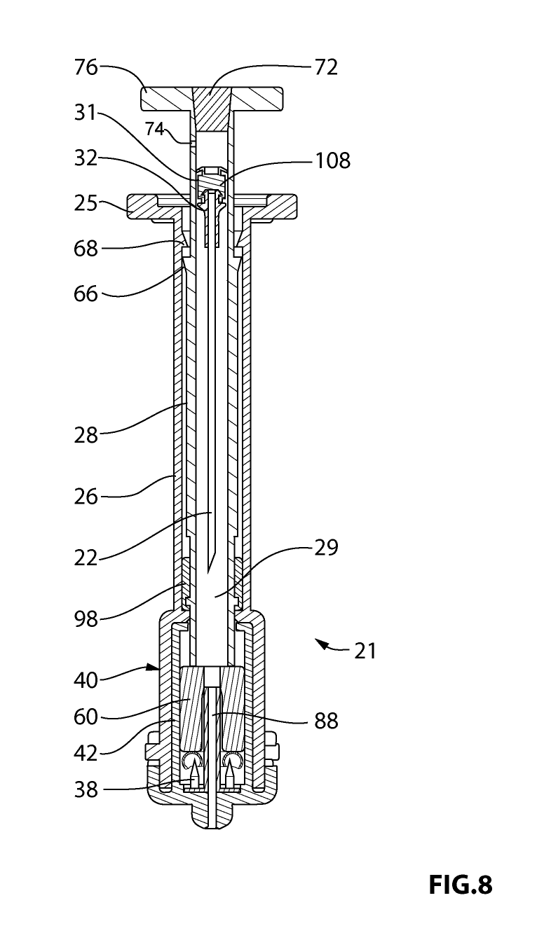

In the illustrated embodiment, a perforator assembly 80 includes a base 82 on which puncture lances 38 are mounted and an elongate central neck 84 projecting proximally from the base 82. Needle 22 extends through central neck 84. In some embodiments, base 82, puncture lances 38, and central neck 84 may be machined as a single piece, or may be separately manufactured and then joined in any suitable manner, for example by adhesives, welding, or the like. Base 82 may be secured to a perforator mount 86 at the distal end of modular retractable needle assembly 20. Preferably, the dimensions and materials of base 82 and perforator mount 86 are such that base 82 mates tightly with perforator mount 86, thereby impeding leakage of gases or other fluids from gas release chamber 36. Perforator assembly 80 includes a needle aperture 88 (FIG. 8). In some embodiments, needle aperture 88 is dimensioned to firmly hold needle 22 during the injection phase. In some embodiments, needle aperture 88 sealingly engages with needle 22. Needle membrane 56 may be secured to cover needle aperture 88.

In the illustrated embodiment, perforator assembly 80 and engagement ring 60 are axially positioned so that a proximal portion of neck 84 extends within the central opening 62 of engagement ring 60. The distal edge of neck 84 contacts needle header 32. Annular gas release cell 34 extends around neck 84.

In the illustrated embodiment as best seen in FIG. 3A, needle header 32 has a downstream hollow cylindrical body 90, a collar 92, and an upstream hollow end knob 94. Needle 22 is crimped in, cemented to, or otherwise securely fixed within body 90 of needle header 32. In the illustrated embodiment, needle header 32 is positioned within central opening 62 of engagement ring 60. In some embodiments, needle header 32 is positioned within modular assembly 20 such that knob 94 is approximately flush with the upstream end of engagement ring 60. Hollow cylindrical body 90 of needle header 32 is inserted into central opening 62 of engagement ring 60 on the proximal side thereof. Needle header 32, including collar 92, is inserted within central opening 62 with a fit approaching a snug fit (i.e. a tight fit). Needle header 32 sealingly engages with engagement ring 60 to prevent leakage of medicament or other fluids between needle header 32 and engagement ring 60.

In some embodiments, the proximal tip of needle header 32 is alternatively positioned above engagement ring 60, such that hollow end knob 94 projects in the proximal direction from engagement ring 60. In such embodiments, corresponding adjustments can be made to recess needle port seal 31 slightly further in the proximal direction within plunger 28. Embodiments wherein needle header 32 is initially positioned within engagement ring 60 as described above may reduce the likelihood that needle header 32 may be accidentally dislodged prior to use, for example due to forces that may be applied in the course of shipping or handling modular retractable needle assembly 20.

To facilitate retraction of needle 22, plunger 28 includes a narrowed distal plunger neck 96 (FIG. 5). Narrowed distal plunger neck 96 includes at its distal end a needle port seal 31. Needle port seal 31 is frictionally and sealingly engaged within the inner surface of distal plunger neck 96 with a snug fit that prevents movement of needle port seal 31 in response to a loading force or an injection force, but that permits movement of needle port seal 31 in the proximal direction within retraction lumen 29 under pressure applied by the rupture of gas release cell 34. Needle port seal 31 also prevents medicament from entering retraction lumen 29 when syringe assembly 21 is in use.

Needle port seal 31 should be positioned within distal plunger neck 96 so that needle port seal 31 engages with needle header 32 at or just before the end of the application of an injection force by a user, or at the start of the application of a post-injection force to plunger 28 by a user. In the illustrated embodiment, needle port seal 31 is mounted within distal plunger neck 96 so that the distal end of needle port seal 31 is flush with the distal portion of distal plunger neck 96. Needle port seal 31 is shaped and configured to engage with needle header 32 without being obstructed by engagement ring 60. In the illustrated embodiment, needle port seal 31 includes a grasping ledge 35 that extends radially inwardly from the distal end of needle port seal to grasp knob 94 of needle header 32.

The diameter of the outer portion of needle port seal 31 is selected so that needle port seal 31 frictionally engages the inner surface of distal plunger neck 96 and of retraction lumen 29. At least the outermost circumferential portion of needle port seal 31 that engages with these surfaces may be made from resilient material, so that needle port seal 31 may expand in diameter to fill the full cross-section of retraction lumen 29 during retraction of needle 22. In the illustrated embodiment, needle port seal 31 includes a sealing portion 33 made from a resilient material that sealingly engages with the walls of retraction lumen 29 to facilitate gas-driven retraction of needle 22.

In some embodiments, retraction lumen 29 may be tapered, such that the proximal portion of retraction lumen 29 has a larger diameter than the distal portion of retraction lumen 29. In such embodiments, the resilient outermost circumferential portion of needle port seal 31 expands as needle port seal 31 moves proximally within retraction lumen 29, to maintain sealing engagement within retraction lumen 29.

To facilitate injection of medicament from medicament chamber 30, a hollow deformable plunger seal 98 is provided on plunger 28. Plunger seal 98 surrounds distal plunger neck 96 with a tight fit. Plunger seal 98 may be constrained from axial movement during normal use of syringe assembly 21 by engagement of a radially outwardly extending collar 100 on distal plunger neck 96 with an annular recess 102 formed on the inside surface of plunger seal 98, or in any other suitable manner. The fit, dimensions and material used for plunger seal 98 are selected so that plunger seal 98 slidingly but sealingly engages the inner wall of barrel 26 with some resistance.

In some embodiments, needle port seal 31 is integrally formed with plunger seal 98. In such embodiments, a thin skin of material (not shown) is provided between needle port seal 31 and plunger seal 98. The thin skin of material is sufficiently strong to withstand the application of both a loading force and an injection force to syringe assembly 21 and prevent leakage of medicament therethrough. However, the thin skin of material is sufficiently weak that it is broken by the distal force applied when gas is release cell 34 is ruptured, allowing needle port seal 31 to be retracted within retraction lumen 29, along with needle header 32 and needle 22.

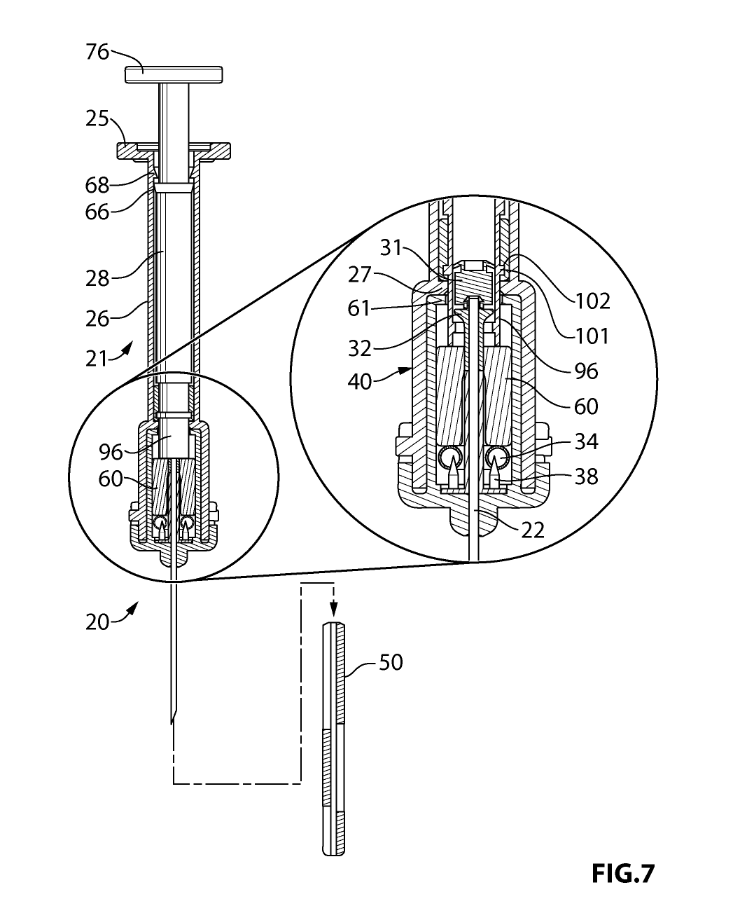

Internal radial wall 27 and/or internal radial wall 61 define an opening that is dimensioned so that distal plunger neck 96 can pass therethrough, but so that plunger seal 98 cannot. Thus, movement of plunger seal 98 in the distal direction is stopped when seal 98 abuttingly engages radial wall 27. Application of a post-injection force thus causes distal plunger neck 96 to penetrate into modular retractable needle assembly 20 through opening 64, while plunger seal 98 is forced slidingly in the proximal direction along distal plunger neck 96. In the illustrated embodiment, engagement of plunger seal 98 with a radially extending shoulder 104 (FIG. 2) formed where the main body 106 of plunger 28 meets distal plunger neck 96 prevents further movement of plunger seal 98 in the proximal direction relative to plunger 28 (FIG. 7).

In use, a user may select a syringe 24 of the desired volume and affix modular needle assembly 20 thereto by engaging securing projection 48 on the syringe barrel 26 with L-shaped slot 46 on securing jacket 45 of the assembly 20. The user can twist assembly 20 relative to syringe 24 so that securing projection 48 slides past angled locking protrusion 49 in L-shaped slot 46 in the downstream direction. Assembly 20 is thus securely coupled to syringe 24. Needle cover 50 may be twisted off or otherwise removed in any suitable manner to expose needle 22.

Downstream force is applied by a user to the upstream plunger end flange 25 and/or to plunger plug 72 to eject air out of medicament chamber 30, if necessary. When nearly all of the air has been forced out of medicament chamber 30, but before plunger verge 66 engages with syringe barrel lock 68, the tip 52 of needle 22 can be submerged in liquid medicament contained in a supply vial, which may be of a conventional type.

Medicament or other liquid for injection is drawn into medicament chamber 30 by withdrawing plunger 28 proximally relative to syringe barrel 26 in the same manner as a conventional syringe. After medicament chamber 30 has been filled with the desired volume of medicament, air may be removed in the conventional manner by inverting syringe assembly 21 so that needle 22 is pointing upwardly, tapping syringe 24 to displace any air therewithin and allowing the air to float above the medicament, and applying a distally-directed force to the plunger 28 so that residual air is forced out through needle 22.

Needle 22 is positioned at an injection site of a subject in the conventional manner. Medicament can be discharged from chamber 30 by applying a distally-directed force on plunger end flange 76 and/or plunger plug 72 in a conventional manner, thus causing plunger seal 98 to exert a distal biasing pressure on the medicament contained in chamber 30. The distally-directed biasing pressure is sufficient to force medicament through needle 22. However, the pressure is not sufficient to overcome the frictional force securing engagement ring 60 to the inner wall of cylindrical axial extension 42, nor is the corresponding upstream pressure on the tip of plunger 28 sufficient to overcome the frictional force between needle carrier 31 and distal plunger neck 96.

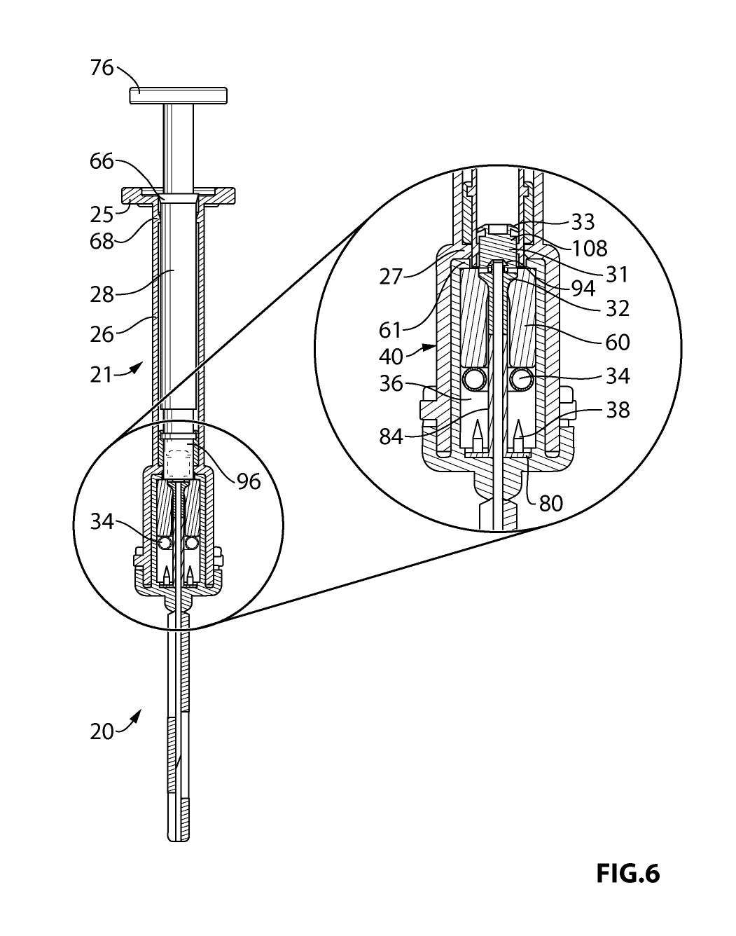

With reference to FIG. 6, after all or substantially all of the medicament has been injected into a subject, a user continues to apply force, now a post-injection force, in the distal direction against plunger 28. Movement of distal plunger neck 96 in the distal direction past internal radial wall 27 causes distal plunger neck 96 to impinge on engagement ring 60. Needle port seal 31 is forced onto hollow end knob 94 of needle header 32, thereby sealing needle header 32 so that no medicament or other fluids may be passed through needle 22. Engagement of needle port seal 31 with hollow end knob 94 forms needle retraction assembly 108 (FIG. 8), which includes needle port seal 31, needle header 32, and needle 22. Further motion of plunger seal 98 in the distal direction is prevented by internal radial wall 27.

Engagement of needle header 32 with central neck 84 of perforator assembly 80 prevents movement of needle retraction assembly 108 in the distal direction. Continued application of a post-injection force causes distal plunger neck 96 to move axially past internal radial wall 27 and to impinge on engagement ring 60. The post-injection force overcomes the frictional engagement between engagement ring 60 and cylindrical axial extension 42, causing engagement ring 60 to move in the distal direction (FIG. 7). Continued motion of engagement ring 60 also forces gas release cell 34 in the distal direction, causing gas release cell 34 to impinge on puncture lances 38. Gas release cell 34 is ruptured, thereby releasing propellant into gas release chamber 36.

The released propellant remains under pressure within the confines of the gas release chamber 36, and therefore a proximal force is applied against needle retraction assembly 108. In embodiments in which the compressed propellant comprises a liquid phase, the propellant will transition to the gas phase when gas release cell 34 is ruptured. The proximal force applied by the released gas is sufficiently strong to overcome the frictional force securing the needle header 32 within the central opening 62 of engagement ring 60, and also to overcome the frictional force securing needle port seal 31 within distal plunger neck 96. The proximally-directed biasing pressure causes needle retraction assembly 108 to slide proximally into retraction lumen 29, thus retracting needle 22 inside retraction lumen 29 (FIG. 8). The volume and pressure of propellant in gas release cell 34 should be sufficient to retract the full length of needle 22 inside retraction lumen 29.

In some embodiments, syringe 24 may initially be coupled to a needle assembly including a non-retractable needle of a relatively larger gauge than would be used to inject medicament into a patient. Syringe 24 may be filled with medicament using the larger gauge needle in the manner described above. The non-retractable needle assembly may then be removed from syringe 24, and syringe 24 may be coupled to a modular retractable needle assembly 20 in the manner described above. Using a larger gauge needle to fill syringe 24 may facilitate faster and/or easier loading of syringe 24.

Suitable materials for the manufacture of syringe assembly 21 may be selected by one skilled in the art. For example, syringe barrel 26 and plunger 28 may be made from a plastic material, such as appropriate polymers or copolymers. Plunger seal 98 may be made from any suitable material, for example elastomers or rubber. In some embodiments, plunger seal 98 may be a self-lubricating seal. In some embodiments, syringe barrel 26 and/or plunger seal 98 may be treated with a medical grade lubricant. Needle 22 may be made of medical grade needle tubing. The compressed propellant used in gas release cell 34 may be any suitable propellant, for example a pharmaceutical-grade hydrofluorocarbon such as 1,1,1,2-tetrafluoroethane or medical-grade nitrogen. Suitable materials for manufacture of gas release cell 34 include suitable polymers such as, for example, nylon, polyethylene, polypropylene, polystyrene or the like, or suitable copolymers thereof. Components may be sterilized prior to packaging in any suitable manner, for example with e-beam radiation, y-radiation, or ethylene oxide (EtO) gas. The materials selected for manufacture of syringe assembly 21 should be compatible with the medicament to be administered to the subject.

In some embodiments, modular needle assembly 20 and portions of syringe 24 may be manufactured to a standard size and configuration to permit assembly 20 to be coupled to different types of syringes having a plurality of different sizes. This permits a user to choose an appropriate size of needle (e.g. a longer or shorter needle, or a needle of higher or lower gauge) or an appropriate size or volume of syringe, depending on the particular task to be accomplished.

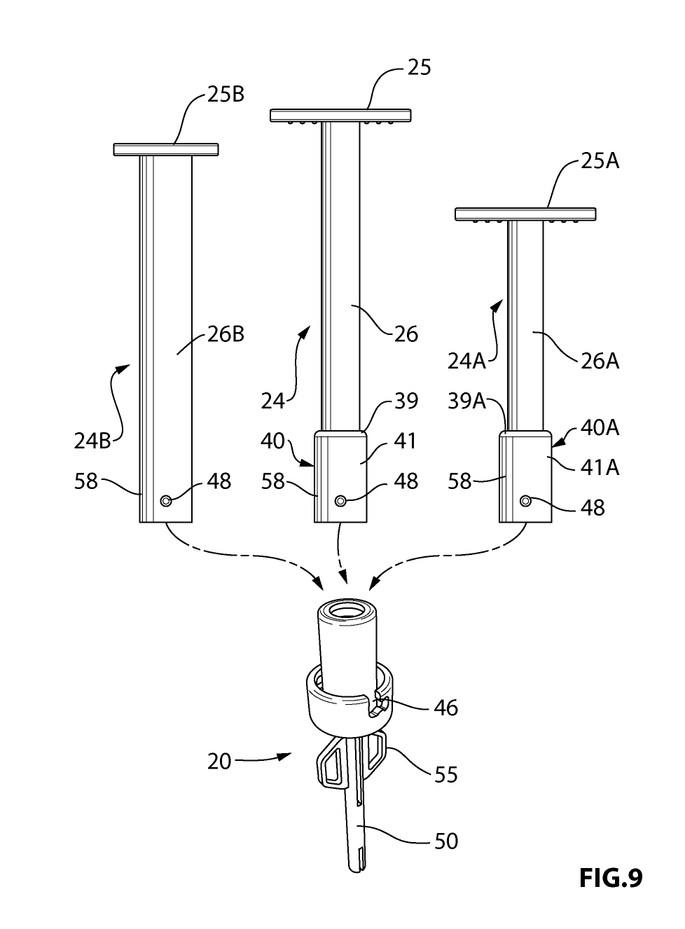

For example, with reference to FIG. 9, a plurality of syringes 24, 24A and 24B are illustrated. Syringe 24 has a barrel 26 with a distal swedge 40. Swedge 40 is provided with a pair of external securing projections 48 for engaging with L-shaped slots 46 on modular retractable needle assembly 20. Swedge 40 provides the mating receptacle 58 of syringe 24. Syringe 24 is also provided with a proximal flange 25.

Syringe 24A is generally similar to syringe 24 but has a smaller overall volume. Syringe 24A has a barrel 26A that is of smaller diameter than barrel 26 of syringe 24. The overall length of syringe 24A is illustrated as being shorter than the length of syringe 24, although the length of syringe 24A need not be shorter than syringe 24 and could be the same or longer. Like syringe 24, syringe 24A is provided with a distal swedge 40A. The interior surface of swedge 40A provides the mating receptacle 58 of syringe 24A. The overall internal and external dimensions of mating receptacle 58 of syringe 24A are the same as mating receptacle 58 of syringe 24. That is, the internal and external diameter of the mating receptacle 58 defined by swedge 40A is the same as the internal and external diameter of the mating receptacle 58 defined by swedge 40. Because barrel 26A is of narrower diameter than barrel 26, proximal wall portion 39A of swedge 40A is slightly longer than proximal wall portion 39 of syringe 24. Swedge 40A is provided with a pair of securing projections 48 for engaging with L-shaped slots 46 of modular retractable needle assembly 20. Thus, even though syringe 24A is of a smaller overall volume than syringe 24, syringe 24A can be coupled to the same modular retractable needle assembly 20.

Syringe 24B is generally similar to syringe 24 but has a larger overall volume. Syringe 24B has a barrel 26B that is of larger diameter than barrel 26 of syringe 24. The overall length of syringe 24B is illustrated as being smaller than syringe 24, but could also be the same or larger. Unlike syringes 24 and 24A, syringe 24B is not provided with a distal swedge 40. Rather, mating receptacle 58 is provided by the distal tip of syringe 24B. The overall internal and external dimensions of mating receptacle 58 of syringe 24B are the same as mating receptacles 58 of syringes 24 and 24A. The diameter of syringe barrel 26B is the same as the diameter of swedges 40 and 40A of syringes 24 and 24A, respectively. Thus, syringe 24B does not require a swedge to provide a mating receptacle 58 having the same dimensions as the mating receptacles 58 of syringes 24 and 24A. The distal end of syringe barrel 26B is also provided with a pair of securing projections 48 for engaging with L-shaped slots 46 of modular retractable needle assembly 20. Thus, even though syringe 24B is of a larger overall volume than syringes 24 and 24A, syringe 24B can be coupled to the same modular retractable needle assembly 20 as syringes 24 and 24A.

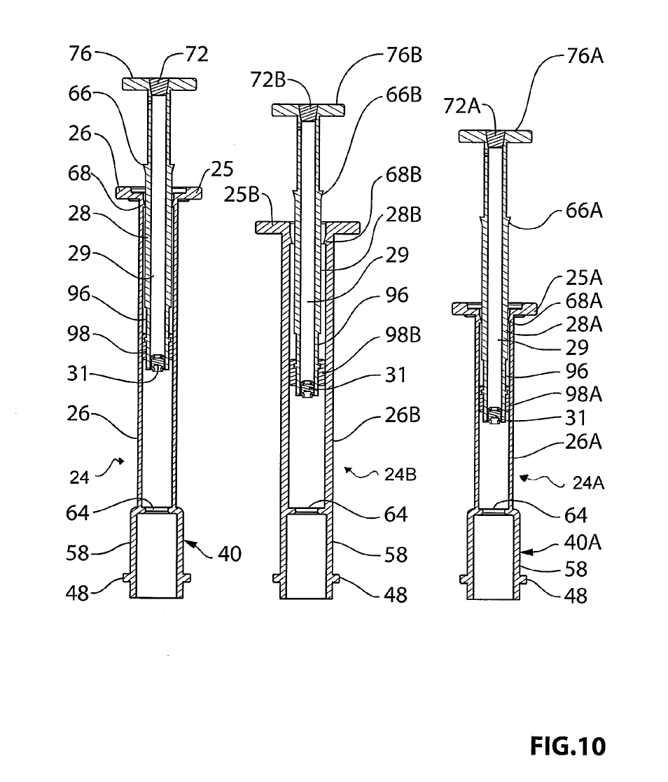

With reference to FIG. 10, syringes 24, 24A and 24B are shown in sectional view with plungers 28, 28A and 28B sealingly engaged therein. The mating receptacle 58 of all three syringes has the same dimensions, to receive cylindrical axial extension 42 of a single modular retractable needle assembly 20 as described above. The distal plunger neck 96 of all three plungers 28, 28A and 28B has the same dimensions, so that a post-injection force applied to each syringe will displace engagement ring 60 of modular retractable needle assembly 20 in the distal direction as described above.

In the illustrated embodiment, the interior diameter of retraction lumen 29 of all of plungers 28, 28A and 28B is the same, and each of the retraction lumens 29 are of sufficient length to receive the entire length of needle 22 when needle 22 is retracted. However, the exterior diameter of plunger 28A is smaller than that of plunger 28, to allow plunger 28A to fit in sliding engagement within the narrower barrel 26A. The exterior diameter of plunger 28B is larger than that of plunger 28, to allow plunger 28B to be slidingly received within syringe 24B without rattling therewithin. Similarly, plunger seal 98B has a diameter larger than plunger seal 98 to facilitate sealing engagement with the interior surface of syringe 24B, while plunger seal 98A has a diameter smaller than plunger seal 98 to facilitate sliding engagement of plunger seal 98A within syringe 24A.

In the illustrated embodiment, the needle port seals 31 of all three syringes 24, 24A and 24B have the same dimensions, to engage with the needle header 32 of modular retractable needle assembly 20. The internal diameter of the plunger lumens 29, 29A and 29B are also the same, to engage with needle port seal 31.

In some embodiments, the internal diameter of the plunger lumens 29 of the differently sized syringes may be varied. In such embodiments, corresponding changes in the external diameter of needle port seal 31 are made so that needle port seal 31 can maintain sealing engagement with plunger lumen 29 while still being sized and shaped to engage with needle header 32. Thus, portions of needle port seal 31 that interact with needle header 32, such as grasping ledge 35, are configured to be of a consistent size to be able to securely grasp needle header 32, but the dimensions of other portions of needle port seal 31, for example the portions of needle port seal 31 that engage with plunger lumen 29 (e.g. gripping arms 33), may be varied if it is desired to vary the dimensions of the plunger lumen across syringes having different sizes. In embodiments in which the dimensions of the plunger are varied across syringes having different sizes, the internal and external diameter of distal plunger neck 96 should be kept approximately the same, to ensure proper interaction with engagement ring 60 and needle header 32 of modular retractable needle assembly 20.

Other than the differences in the dimensions of some components as noted above, the construction and operation of plungers 28A and 28B is substantially similar to plunger 28. Plungers 28A and 28B may also include plunger lock verge 66A and 66B, respectively, which engage with a corresponding syringe barrel lock 68A or 68B on syringe 24A or 24B, respectively, in the same manner as described above with reference to syringe 24. Plungers 28A and 28B may optionally also include an opening at their proximal end, and a plunger plug 72A, 72B for insertion into the opening as described above for plunger plug 72 of syringe 24.

In the foregoing manner, a single modular needle assembly 20 may be fitted to a syringe of any desired volume, for example 1 cc, 1.5 cc, 2 cc, 2.5 cc, 3 cc, 5 cc, 10 cc, 25 cc, 50 cc, or the like, by varying the diameter of the syringe barrel and providing a swedge of consistent dimensions for engaging with modular needle assembly 20.

In some embodiments, a modular retractable needle assembly 20 of one size may be provided for engagement with a plurality of syringes with a predetermined range of volumes, for example syringe sizes from 1 cc to 5 cc and any size therebetween, e.g. 1.5 cc, 2 cc, 2.5 cc, 3 cc, or 4 cc. A second modular retractable needle assembly 20 of a somewhat larger size, including a larger size of gas release cell 34 containing a larger volume of compressed propellant (which may be in liquid form), may be provided for engagement with syringes having a different range of sizes, for example 10 cc to 25 cc and any size therebetween, e.g. 15 cc or 20 cc. In some embodiments, a third modular retractable needle assembly 20 of an even larger size, including a larger size of gas release cell 34, may be provided for engagement with syringes of larger size, for example 50 cc to 100 cc and any size therebetween.

As described above, FIG. 9 shows three different sizes of syringe, 24, 24A, and 24B that are configured to be engaged with a single modular retractable needle assembly 20. In some embodiments, syringe 24B may be a 5 cc syringe, syringe 24 may be a 3 cc syringe, and syringe 24A may be a 1 cc syringe.

In some embodiments, modular retractable needle assembly 20 is provided as part of a kit including one or more modular retractable needle assemblies 20 and one or more of a plurality of different sizes of syringe suitable for engagement with that particular size of retractable modular needle assembly 20. For example and without limitation, one or more modular retractable needle assemblies 20 suitable for engagement with syringes having a size of 1 cc, 3 cc or 5 cc may be provided as part of a kit with a plurality of retractable syringes of 1 cc, 3 cc and 5 cc in size. Each of the 1 cc, 3 cc and 5 cc syringes has a mating receptacle 58 having dimensions suitable for engagement with the same modular retractable needle assembly 20.

In some embodiments, modular retractable needle assembly 20 and/or syringe 24 may be colour-coded to indicate various parameters. For example, modular retractable needle assembly 20 may be colour coded to indicate the length and/or gauge of needle 22 present therein. Alternatively, modular retractable needle assembly 20 may be colour coded to indicate the size range of syringes with which that particular assembly 20 may be used. Alternatively or additionally, syringes 24 may be colour coded to indicate which particular modular retractable needle assembly 20 may be used with that size of syringe. In some such embodiments, the colour of modular retractable needle assembly 20 may be the same colour as the range of syringes 24 with which that particular assembly 20 may be used.

In some embodiments, modular retractable needle assembly 20 and/or syringe 24 are colour coded in a particular colour to differentiate these components from conventional syringes and needles (i.e. to identify assembly 20 and syringe 24 as components of a retractable syringe assembly).

In some embodiments, syringe 24 is a prefilled syringe, i.e. syringe 24 has been filled with a predetermined quantity of a specified medicament. In use, prefilled syringe 24 does not need to be loaded with medicament, but can simply be coupled to modular retractable needle assembly 20 in the manner described above and used.

In some embodiments, a non-retractable needle assembly having a mating surface of suitable dimensions to engage with mating receptacle 58 of syringe 24 may be used to load medicament into syringe 24, and a second, modular retractable needle assembly 20 may be used to inject the medicament into a subject. In some such embodiments, the non-retractable needle assembly may have a needle of higher gauge than the modular retractable needle assembly 20 that is used.

While a number of exemplary aspects and embodiments have been discussed above, those of skill in the art will recognize certain modifications, permutations, additions and sub-combinations thereof. Without limitation, such modifications may include:
while cylindrical axial extension 42 has been described as being of sufficient diameter to sealingly engage with interior surfaces of syringe barrel 26, cylindrical axial extension 42 could be of a relatively narrower diameter, and a circumferential protrusion on extension 42 could sealingly engage with the interior surface of syringe barrel 26;
the relative orientation of puncture lances 38 and gas release cell 34 could be reversed, so that puncture lances 38 are provided on the distal side of engagement ring 60;
internal radial wall 61 on modular retractable needle assembly 20 and/or internal radial wall 27 of syringe 24 may be omitted, and further movement of plunger seal 98 in the distal direction upon application of a post-injection force may be prevented by engagement of plunger seal 98 with the proximal end of cylindrical axial extension 42, or not prevented at all;
the shape of the mating surface of assembly 20 and of mating receptacle 58 of syringe 24 could be varied, so long as the shape of the mating receptacle 58 is complementary to the shape of the mating surface and so long as the shape of the internal components of the syringe assembly 21 allows for retraction of needle 22 in response to the application of a post-injection force;
while syringe 24A has been described as having a smaller volume than syringes 24 and 24B, in some embodiments syringe 24A could have a larger volume than syringe 24 and/or syringe 24B; and
while syringe 24B has been described as having a larger volume than syringes 24 and 24A, in some embodiments syringe 24B could have a smaller volume than syringe 24 and/or syringe 24A.
   Accordingly, the scope of the claims should not be limited by the preferred embodiments set forth in the description, but should be given the broadest interpretation consistent with the specification as a whole.

1. A modular gas-actuated retractable syringe assembly comprising:
a syringe for receiving a retractable needle, the syringe comprising a barrel, a medicament chamber within the barrel, and a plunger, the plunger being slidably disposed within the syringe barrel to displace medicament from the medicament chamber; and
a modular gas-actuated retractable needle assembly detachably engageable with the syringe so that the needle is placed in fluid communication with the medicament chamber to administer the medicament, the modular gas-actuated retractable needle assembly comprising a gas release cell and a gas cell puncturer disposed within a gas release chamber of the modular gas-actuated retractable needle assembly,
wherein the application of a post-injection force to the plunger by a user causes the gas cell puncturer to rupture the gas release cell, and the pressure of the released gas within the gas release chamber forces the needle to be retracted within the syringe.

2. A modular gas-actuated retractable syringe assembly as defined in claim 1, wherein the needle comprises a needle header at a proximal end of the needle and the plunger comprises a needle port seal at a distal end of the plunger, and wherein the needle port seal is engageable with the needle header to provide a needle carrier upon the application of the post-injection force.

3. A modular gas-actuated retractable syringe assembly as defined in claim 1, wherein the plunger comprises a retraction lumen and the needle port seal is sealingly engaged within the retraction lumen.

4. A modular gas-actuated retractable syringe assembly as defined in claim 1, comprising a distal plunger neck of relatively narrower diameter than the rest of the plunger neck and an internal radial wall at a distal end of the syringe through which the distal plunger neck passes upon application of a post-injection force by a user.

5. A modular gas-actuated retractable syringe assembly as defined in claim 1, comprising a distal plunger neck of relatively narrower diameter than the rest of the plunger neck and an internal radial wall at a proximal end of the modular gas-actuated retractable needle assembly through which the plunger neck passes upon application of a post-injection force by a user.

6. A modular gas-actuated retractable syringe assembly as defined in claim 1, wherein the syringe comprises a swedge at its distal end.

7. A modular gas-actuated retractable syringe assembly as defined in claim 6, wherein the swedge comprises a proximal wall portion extending radially outwardly from the syringe barrel and a distally projecting cylindrical extension of wider diameter than the rest of the syringe barrel extending from the proximal wall portion.

8. A modular gas-actuated retractable syringe assembly as defined in claim 1, wherein a distal end of the syringe comprises a securing projection and wherein the modular gas-actuated retractable needle assembly comprises an L-shaped slot positioned and configured to engage with the securing projection when in a secured position to prevent relative movement of the syringe and the modular retractable needle assembly in an axial direction.

9. A modular gas-actuated retractable syringe assembly as defined in claim 8, wherein the L-shaped slot is provided on an axial extension of the modular gas-actuated retractable needle assembly that is radially spaced apart from a mating surface of the modular gas-actuated retractable needle assembly, the mating surface being shaped and configured for engagement with the distal end of the syringe.

10. A modular gas-actuated retractable syringe assembly as defined in claim 9, wherein the mating surface of the modular gas-actuated retractable needle assembly engages with an interior surface of the distal end of the syringe, the securing projection extends radially outwardly from the syringe, and the axial extension is spaced radially outwardly from the mating surface of the modular gas-actuated retractable needle assembly.

11. A modular gas-actuated retractable syringe assembly as defined in claim 10, comprising an O-ring seal sealingly engaged between a distal end of the syringe and the modular gas-actuated retractable needle assembly.

12. A modular gas-actuated retractable syringe assembly as defined in claim 8, comprising a locking element to prevent the modular retractable needle assembly from being removed from the syringe after the syringe assembly has been assembled.

13. A modular gas-actuated retractable syringe assembly as defined in claim 12, wherein the locking element comprises an one-way projection disposed within the L-shaped slot, the one-way projection being sized and configured to allow the securing projection to slide past the one-way projection into a locked configuration.

14. A modular gas-actuated retractable syringe assembly as defined in claim 13, wherein the one-way projection comprises a first surface past which the securing projection can slide into the locked configuration, and a second surface that prevents movement of the securing projection out of the locked configuration.

15. A modular gas-actuated retractable syringe assembly as defined in claim 14, wherein the second surface comprises a substantially axially extending surface.

16. (canceled)

17. A modular gas-actuated retractable syringe assembly as defined in claim 1, comprising a vent hole in the plunger.

18. A modular gas-actuated retractable syringe assembly as defined in claim 2, comprising a plunger seal, wherein the needle port seal is integrally formed with the plunger seal, wherein a thin skin of material joins the needle port seal to the plunger seal, and wherein the thin skin is strong enough to remain intact during application of a loading force and an injection force, but weak enough to break when the gas release cell is ruptured.

19. A detachable modular gas-actuated retractable needle assembly for coupling to a syringe so that the needle is placed in fluid communication with a medicament chamber of the syringe to administer medicament, the syringe having a plunger for administering medicament with a retraction lumen therein and a needle carrier for engaging with and retracting the needle under gas pressure, the assembly comprising:
a mating surface for engaging with the syringe;
a needle releasably secured within the assembly;
a gas release chamber defined between a distal portion of the assembly and the needle;
a gas release cell disposed within the gas release chamber; and
a gas cell perforator disposed within the gas release chamber and operable in response to the application of a post-injection force to rupture the gas release cell,
wherein, in use, a post-injection force applied by a user to the plunger couples the needle to the needle carrier and causes the gas cell perforator to puncture the gas release cell.

20-28. (canceled)

29. A syringe for use with a modular gas-actuated retractable needle assembly having a needle with a needle header at a proximal end of the needle, a gas release cell and a gas release trigger mechanism with a gas release chamber of the modular gas-actuated retractable needle assembly, the syringe comprising:
a barrel having a first diameter;
a distal swedge having a diameter greater than the first diameter;
a plunger axially slidable and sealingly engaged within the barrel of the syringe, the plunger having a retraction lumen therein;
a needle port seal initially sealingly engaged within the plunger at a distal end of the retraction lumen; and
a medicament chamber defined within the barrel distally of the plunger, the medicament chamber being configured to be placed in fluid communication with the needle when the syringe is assembled with the modular gas-actuated retractable needle assembly;
wherein, in use when assembled with the modular gas-actuated retractable needle assembly upon the application of a post-injection force to the plunger by a user, the needle port seal engages with the needle header and the gas release trigger mechanism ruptures the gas release cell so that the needle port seal is displaced axially in the proximal direction with respect to the retraction lumen to retract the needle within the retraction lumen.

30. A kit comprising a plurality of syringes of different volumes for engagement to a single modular retractable needle assembly, wherein:
each of the syringes comprises:
a barrel;
a plunger axially slidable and sealingly engaged within the barrel of the syringe for administering medicament, the plunger having a retraction lumen therein;
a needle port seal sealingly engaged within the plunger at a distal end of the retraction lumen; and
a medicament chamber defined within the barrel distally of the plunger; and
the single modular retractable needle assembly comprises:
a mating surface for engaging with the syringe;
a needle releasably secured within the assembly for being placed in fluid communication with the medicament chamber;
a gas release chamber defined between a distal portion of the assembly and the needle;
a gas release cell disposed within the gas release chamber; and
a gas cell perforator disposed within the gas release chamber and operable in response to the application of a post-injection force to rupture the gas release cell,
each of the syringes being detachably engageable with the single modular retractable needle assembly,
wherein, in use, application of a post-injection force to the plunger by a user couples the needle to the needle carrier and causes the gas cell perforator to puncture the gas release cell.

31. A kit as defined in claim 30, wherein at least one of the syringes comprises a distal swedge.

32-35. (canceled)
